(19) United States
(12) Patent Application Publication
(10) Pub. No.: US 2020/0054859 A1
(43) Pub. Date: Feb. 20, 2020

(54) Intra-Balloon Deflection Mechanism for Enhanced Balloon Maneuverability

(71) Applicant: Biosense Webster (Israel) Ltd., Yokneam (IL)

(72) Inventors: Joseph Thomas Keyes, Sierra Madre, CA (US); Kevin Justin Herrera, West Covina, CA (US); Christopher Thomas Beeckler, Brea, CA (US)

(21) Appl. No.: 16/103,793

(22) Filed: Aug. 14, 2018

(51) Int. Cl. A61M 25/01; A61B 18/14; A61M 25/09

(52) U.S. Cl. CPC A61M 25/0147; A61B 2018/0022; A61M 25/09; A61B 18/1492

(57) ABSTRACT

A medical instrument includes an expandable balloon, an intra-balloon deflection assembly, and one or more puller wires. The expandable balloon is coupled to a distal end of a shaft for insertion into a body of a patient. The intra-balloon deflection assembly is also coupled to the distal end of the shaft, and includes a distal section of the shaft which extends partially through the expandable balloon and includes (i) an elastic element coupled to a proximal side of the expandable balloon and configured to be bent relative to a longitudinal axis of the shaft, and (ii) a rigid element coupled to a distal side of the expandable balloon. The one or more puller wires are coupled to the rigid element and are configured to bend the elastic element, thereby deflecting the expandable balloon.

### FIELD OF THE INVENTION

The present invention relates generally to medical probes, and particularly to balloon catheters.

### BACKGROUND OF THE INVENTION

Various catheters employ steering mechanisms so as to maneuver their distal end. For example, U.S. Patent Application Publication 2015/0141982 describes a catheter that has a balloon electrode assembly with at least one compliant balloon member and at least one electrode carried on an outer surface of the balloon member for accomplishing circumferential sensing or ablation in a tubular region of the heart, including a pulmonary vein or ostium. The catheter may also include an electrode assembly with a tip and/or ring electrode distal of the balloon electrode assembly adapted for focal contact. In an embodiment, a puller wire enables bi-directional deflection of the catheter.

As another example, U.S. Pat. No. 6,585,717 describes deflection mechanisms that are positioned so as to deflect portions of a flexible body, such as a catheter, in more than one direction in a single plane, as well as in more than one plane. The invention allows a distal portion of a catheter to be deflected more than 360 degrees to provide a loop. In an embodiment, a deflection structure of the catheter may be made of polymer, a spring-tempered stainless or super-elastic alloy that when released from a sheath will force the catheter tip to take a shape desired. Tension may be applied to a pull-wire, thereby causing the deflection structure to bend.

U.S. Pat. No. 5,395,327 describes a steering mechanism that includes a steering shaft coupled to a controller which includes a handle and apparatus for manipulating the distal end of the steering shaft. The steering shaft includes a flexible coiled spring having a lead spring fixed in position with respect to a distal end thereof in the distal end of the steering shaft. One or more steering wires is affixed at the distal ends thereof to the lead spring. The steering wires extend through the steering shaft to the controller, and the steering apparatus of the controller is used to place tension on one or both of the steering wires. The attachment of the distal ends of the steering wires to the lead spring may be opposite one another or may be offset for providing greater maneuverability. Tension may be placed on the steering wires by wedges mounted transversely to the controller housing, or by rotation of a shaft mounted transversely to the controller housing, the steering wires being attached to the shaft such that rotation in one direction tenses one steering wire, and rotation in the other direction tenses the other steering wire. Two independently rotatable shafts may be used to separately control the two steering wires.

### SUMMARY OF THE INVENTION

An embodiment of the present invention provides a medical instrument including an expandable balloon, an intra-balloon deflection assembly, and one or more puller wires. The expandable balloon is coupled to a distal end of a shaft for insertion into a body of a patient. The intra-balloon deflection assembly is also coupled to the distal end of the shaft, and includes a distal section of the shaft which extends partially through the expandable balloon and includes (i) an elastic element coupled to a proximal side of the expandable balloon and configured to be bent relative to a longitudinal axis of the shaft, and (ii) a rigid element coupled to a distal side of the expandable balloon. The one or more puller wires are coupled to the rigid element and are configured to bend the elastic element, thereby deflecting the expandable balloon.

In some embodiments, the elastic element includes a spring.

In some embodiments, the rigid element includes a stiffening tube, which is configured to prevent the elastic element from bending over at least part of a length of the elastic element.

In an embodiment, a length of the stiffening tube is configured to determine a bending-location over the elastic element.

There is additionally provided, in accordance with an embodiment of the present invention, a medical method, including inserting into a body of a patient a medical instrument, including (i) an expandable balloon coupled to a distal end of a shaft, and (ii) an intra-balloon deflection assembly coupled to the distal end of the shaft, the intra-balloon deflection assembly including: a distal section of the shaft, which extends partially through the expandable balloon and includes (a) an elastic element coupled to a proximal side of the expandable balloon and configured to be bent relative to a longitudinal axis of the shaft, and (b) a rigid element coupled to a distal side of the expandable balloon. One or more puller wires are coupled to the rigid element and configured to bend the elastic element, thereby deflecting the expandable balloon. The expandable balloon is navigated into an organ of the patient. The expandable balloon is deflected using the intra-balloon deflection assembly, so as to access tissue inside the organ. A medical procedure is performed on tissue using the expandable balloon.

In an embodiment, deflecting the expandable balloon includes deflecting the balloon with a force required to achieve deflection at by at least a right angle, relative to the longitudinal axis of the shaft. In an embodiment, performing the medical procedure includes ablating the tissue.

There is further provided, in accordance with an embodiment of the present invention, a medical instrument, including an expandable balloon, a flexible guidewire lumen, a stiffening tube, and one or more puller wires. The expandable balloon is coupled to a distal section of a shaft for insertion into a body of a patient. The flexible guidewire lumen is surrounded by a coil spring. The stiffening tube is coupled to the flexible guidewire lumen. The one or more puller wires are coupled to the distal section and configured to bend the flexible guidewire lumen, thereby deflecting the expandable balloon.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a balloon catheterization system comprising a deflectable balloon catheter, in accordance with an embodiment of the present invention;

FIG. 2 is a schematic, pictorial illustration of the deflectable balloon catheter of FIG. 1, which comprises an intra-balloon deflection assembly, in accordance with an embodiment of the present invention;

FIGS. 3A and 3B are diagrams that schematically illustrate the deflectable catheter of FIG. 2 in straight and deflected states, in accordance with an embodiment of the present invention; and

FIG. 4 is a flow chart that schematically illustrates a method of balloon treatment using an intra-balloon deflection assembly, in accordance with an embodiment of the present invention.

### DETAILED DESCRIPTION OF EMBODIMENTS

### Overview

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

Embodiments of the present invention that are described and illustrated hereinafter provide a cardiac expandable balloon catheter which comprises an intra-balloon deflection assembly. The expandable balloon is coupled to a distal end of a shaft for insertion into a body of a patient. The disclosed deflection assembly enables a user to sharply deflect (i.e., bend) the balloon relative to its longitudinal axis, being assisted by the natural flexibility (e.g., compliance) of the balloon, so as to bring one or more of the electrodes disposed over the balloon into firm contact with cardiac cavity tissue which is otherwise hard to access.

In some embodiments, the deflection assembly comprises a distal section of the shaft, which extends partially through the expandable balloon and comprises (i) an elastic element, such as a spring or an elastic beam, coupled to a proximal side of the expandable balloon and configured to be bent relative to a longitudinal axis of the shaft, and (ii) a rigid element coupled to a distal side of the expandable balloon. That way, the entire deflection assembly is located inside the balloon.

To establish the intra-balloon deflection functionality, the rigid element is coupled at its proximal end to the elastic element, wherein the relative lengths of the rigid part and the flexible (i.e., elastic) part determines the deflection center (i.e., center point of deflection inside the balloon). Specifically, this arrangement allows the selection of a location over the elastic element where the elastic element bends (i.e., a location over the elastic element from which the intra-balloon deflection assembly is deflected).

Additionally, one or more puller wires are coupled to the rigid element and configured to bend the elastic element, thereby deflecting the expandable balloon. For example, when pulled by the operating physician, the one or more puller wires may deflect the rigid element in one of numerous sideways directions relative to the distal end of the shaft.

In an embodiment, the elastic element spans the entire diameter of the balloon, parallel to a longitudinal axis of the shaft. The rigid element comprises a stiffening tube that rigidly fixes the elastic element over a length covering at least the distal half of the elastic element. This fixation prevents the elastic element from bending at a location over the covered part of the elastic element. The one or more puller wires are attached at a distal end of the stiffening tube. The balloon is coupled at its proximal end to the distal end of the shaft. The distal end of the balloon is coupled to the distal end of the stiffening tube. When pulled with the puller wires, the stiffening tube deflects the balloon about a location over the elastic element inside the balloon.

In an embodiment, the modulus strength of the elastic element is selected based on determining a maximal required pulling force of a puller wire so as to fully deflect the stiffening tube, for example to deflect the balloon by at a right angle relative to the longitudinal axis of the shaft. In another embodiment, the balloon is able to take on sharp deflection angles while maintaining flowing irrigation for tissue cooling during ablation.

The above described arrangement of the various components of the intra-balloon deflection assembly allows the deflection of the balloon closer to the geometrical center of the balloon. Therefore, locations at one or more ablation electrodes of the balloon can, with sharp deflection angles, make contact with tissue located over wide range of angles relative to the balloon. For example, the disclosed intra-balloon deflection assembly allow one or more of the balloon electrodes to contact tissue located directly or indirectly proximal to the balloon when, for example, a user deflects the balloon in an approximate right angle.

In some embodiments, a related balloon treatment method is provided, i.e., which uses the intra-balloon deflection assembly. The method comprises inserting the balloon catheter into a body of a patient and advancing (i.e., navigating) the balloon into a target organ. When the balloon is inside the organ, the physician intra-deflects the balloon, so as to enable the balloon access target tissue. Next, the physician further maneuvers the catheter to establish physical contact between the intra-deflected balloon and target tissue. Once the physician had made the contact, the physician can treat tissue, for example by applying radiofrequency ablation using part of the electrodes that he brought to contact with tissue.

The disclosed intra-balloon deflection mechanism and related balloon treatment method gives a physician access to tissue with a balloon catheter that might otherwise be less accessible, or inaccessible, to treatment, if limited by the simple maneuvers available to catheters not provided with the disclosed mechanism and method. Such maneuverability increases the chances of successful completion of a diagnostic and/or therapeutic invasive cardiac procedure, such as pulmonary vein isolation (PVI) for treatment of Atrial Fibrillation.

### System Description

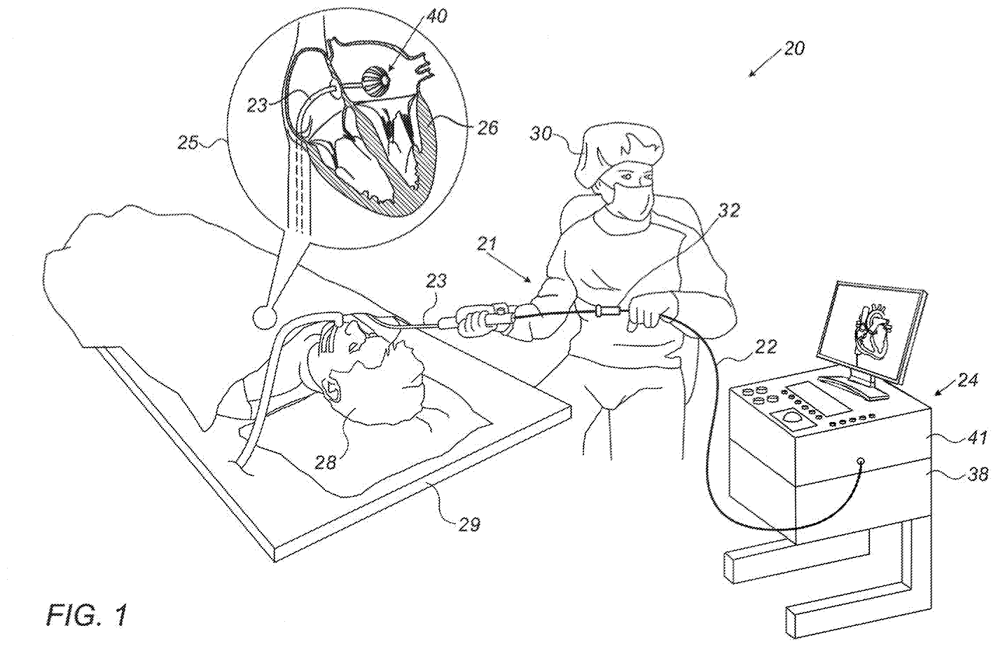

FIG. 1 is a schematic, pictorial illustration of a balloon catheterization system comprising a deflectable balloon catheter 40, in accordance with an embodiment of the present invention. System 20 comprises a catheter 21, wherein a distal end of shaft 22 of the catheter is inserted through a sheath 23 into a heart 26, seen in inset 25, of a patient 28 lying on a table 29. The proximal end of catheter 21 is connected to a control console 24. In the embodiment described herein, catheter 21 may be used for any suitable therapeutic and/or diagnostic purpose, such as electrical sensing, or balloon angioplasty and ablation of tissue in heart 26, among other possible medical usages of expandable balloon catheters.

Physician 30 navigates the distal end of shaft 22 to a target location in heart 26 by manipulating shaft 22 using a manipulator 32 near the proximal end of the catheter and/or deflection from the sheath 23. During the insertion of shaft 22, balloon catheter 40 is maintained in a collapsed configuration by sheath 23. By containing balloon catheter 40 in a collapsed configuration, sheath 23 also serves to minimize vascular trauma along the way to the target location.

Control console 24 comprises a processor 41, typically a general-purpose computer, with a suitable front end and interface circuits 38 for receiving signals from catheter 21, as well as for applying treatment via catheter 21 in heart 26 and for controlling the other components of system 20. Processor 41 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

The example configuration shown in FIG. 1 is chosen purely for the sake of conceptual clarity. The disclosed techniques may similarly be applied using other system components and settings. For example, system 20 may comprise other components and perform non-cardiac treatments.

### Intra-Balloon Deflection Mechanism for Enhanced Balloon Maneuverability

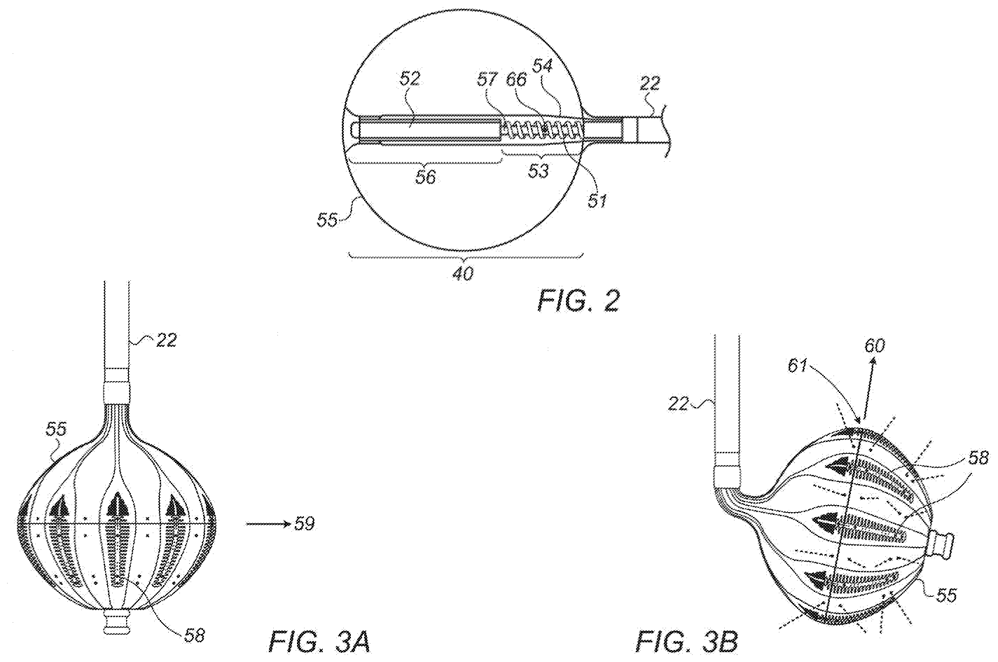

FIG. 2 is a schematic, pictorial illustration of deflectable balloon catheter 40 of FIG. 1, which comprises an intra-balloon deflection assembly, in accordance with an embodiment of the present invention. Balloon catheter 40 is coupled to a distal section 56 of shaft 22 via a coil spring 51, where spring 51 may span the entire length of a balloon 55 to the distal end of the balloon. A portion of spring 51, which envelopes a supporting flexible guidewire lumen 57, runs inside a stiffening tube 52, such that a remaining flexible segment 53 is created (i.e., the flexible element). Two puller wires 54, coupled to stiffening tube 52, enable bi-directional deflection of balloon 55. In an embodiment, the required length of stiffening tube 52 is chosen so as to determine the length of a flexible element 53. The selection causes the bending of spring 51 to occur at location 66 over spring 51. In the exemplified embodiment, balloon 55 deflects about a bending-location 66 over spring 51, which is located over the longitudinal axis of shaft 22. In one example embodiment, bending-location 66 is located at about a sixth of the diameter of balloon 55 distally to the distal-most end of shaft 22 (i.e., distally to the proximal-most end of balloon 55).

While the exemplary embodiments illustrate that the distal section 56 and supporting elastic guidewire lumen 57 of shaft 22 is configured to extend through the balloon, it is within the scope of this invention to have the distal section extends partially into the balloon. Thus, the example illustration shown in FIG. 2 is chosen purely for the sake of conceptual clarity. Any other suitable configuration can be used in alternative embodiments, for example, more than two puller wires 54 may be used to enable multi-directional deflection of balloon 55. The length of stiffening tube 52 may vary, and, correspondingly, bending-location 66 as well. The elastic modulus of spring 51 may vary to determine, for example, the pulling force at which balloon 55 would deflect at approximately a right angle relative to the longitudinal axis of the shaft. In an embodiment, the elastic modulus is in the order of one megapascal (i.e., 10⁶ N/m²). The elastic modulus may also vary to affect the buckle force of the assembly. Moreover, spring 51 itself is provided as an example of an elastic segment. Any flexural component may replace the spring, for example, a flexible beam.

In some embodiments, balloon catheter 40 requires about 8 millimeters of axial travel to elongate (i.e., collapse) balloon 55 sufficiently so that balloon 55 can be easily withdrawn into sheath 23. This requires approximately 4 lbs. of force, or a spring constant of approximately 0.5 lbs./mm (i.e., about 2,000 N/m). Spring can be made of stainless steel, nitinol, beryllium copper, phosphor bronze, or any other similar material with suitable elastic properties.

Moreover, the use of a stiffening tube around a part of an elastic element is also not mandatory. In alternative embodiments, the deflection assembly may comprise any other structure in which the distal part of the section inside the balloon is rigid, and the proximal part of the section inside the balloon is flexible.

It is noted that the balloon can be expanded by any suitable technique such as by mechanical expansion as shown and described in U.S. Pat. No. 9,907,610 (which is incorporated herein by reference), or by a combination of mechanical and hydraulic (via saline fluid flow) expansion techniques.

FIGS. 3A and 3B are photographs of deflectable catheter 40 in straight and deflected states, in accordance with an embodiment of the present invention. As seen, balloon catheter 40 is fitted at the end of shaft 22.

FIG. 3A shows balloon 55 generally aligned parallel to shaft 22. In this configuration, RF electrodes 58 are configured to press against tissue primarily located perpendicularly to shaft 22, i.e., in a radial direction 59. An example of such a commonly occurring configuration is a procedure for pulmonary vein isolation.

FIG. 3B shows balloon 55 deflected proximally (by pulling at least one of the puller wires). The balloon is seen as expanded. As seen, cooling fluid (e.g., saline to cool blood and tissue) flows out of irrigation pores. As further seen, the balloon is bent such that a surface of the balloon at a point 61, which is located approximately on the balloon equator, is directed at a proximal direction 60. Such balloon bending may bring at least one of its radiofrequency electrodes 58 into contact with tissue, which would otherwise be hard to access with another type of ablation balloon.

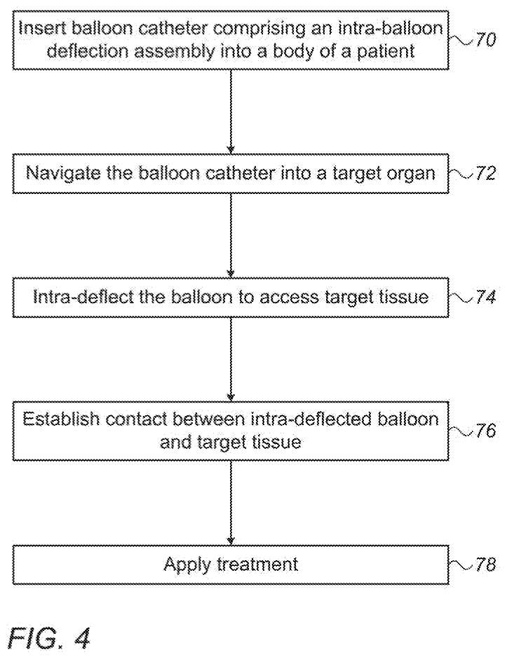

FIG. 4 is a flow chart that schematically illustrates a method of balloon treatment using an intra-balloon deflection assembly, in accordance with an embodiment of the present invention. A treatment begins with physician 30 inserting balloon catheter 40 into a body of a patient, at a balloon insertion step 70. Next, the physician navigates the balloon catheter into a target organ, at a balloon navigation step 72. Next, at a balloon intra-deflection step 74, physician 30 intra-deflects the balloon, so as to enable the balloon access target tissue. Then, physician 30 maneuvers the shaft so as to establish firm contact between, for example, a proximal surface of intra-deflected balloon and target tissue, at a balloon contacting step 76. The physician next treats tissue, at a balloon treatment step 78, for example by radiofrequency ablation using the electrodes brought to contact with tissue.

The example flow chart shown in FIG. 4 is chosen purely for the sake of conceptual clarity. Additional steps, such as the expansion of the balloon and the operation of irrigation, are omitted from the purposely highly simplified flow chart.

Although the embodiments described herein mainly address pulmonary vein isolation, the methods and systems described herein can also be used in other applications, such as in otolaryngology or neurology procedures.

It will thus be appreciated that the embodiments described above are cited by way of example, and that the present invention is not limited to what has been particularly shown and described herein above. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
an expandable balloon, coupled to a distal end of a shaft for insertion into a body of a patient; and
an intra-balloon deflection assembly coupled to the distal end of the shaft, the intra-balloon deflection assembly comprising:
a distal section of the shaft, which extends at least partially through the expandable balloon and comprises (i) an elastic element coupled to a proximal side of the expandable balloon and configured to be bent relative to a longitudinal axis of the shaft, and (ii) a rigid element coupled to a distal side of the expandable balloon; and
one or more puller wires, which are coupled to the rigid element and configured to bend the elastic element, thereby deflecting the expandable balloon.

2. The medical instrument according to claim 1, wherein the elastic element comprises a spring.

3. The medical instrument according to claim 1, wherein the rigid element comprises a stiffening tube, which is configured to prevent the elastic element from bending over at least part of a length of the elastic element.

4. The medical instrument according to claim 3, wherein a length of the stiffening tube is configured to determine a bending-location over the elastic element.

5. A medical method, comprising:
inserting into a body of a patient a medical instrument, comprising (i) an expandable balloon coupled to a distal end of a shaft, and (ii) an intra-balloon deflection assembly coupled to the distal end of the shaft, the intra-balloon deflection assembly comprising:
a distal section of the shaft, which extends partially through the expandable balloon and comprises (a) an elastic element coupled to a proximal side of the expandable balloon and configured to be bent relative to a longitudinal axis of the shaft, and (b) a rigid element coupled to a distal side of the expandable balloon; and
one or more puller wires, which are coupled to the rigid element and configured to bend the elastic element, thereby deflecting the expandable balloon;
navigating the expandable balloon into an organ of the patient;
deflecting the expandable balloon using the intra-balloon deflection assembly, so as to access tissue inside the organ; and
performing a medical procedure on tissue using the expandable balloon.

6. The method according to claim 5, wherein deflecting the expandable balloon comprises deflecting the balloon with a force required to achieve deflection at by at least a right angle, relative to the longitudinal axis of the shaft.

7. The method according to claim 5, wherein performing the medical procedure comprises ablating the tissue.

8. A medical instrument, comprising:
an expandable balloon, coupled to a distal section of a shaft for insertion into a body of a patient;
a flexible guidewire lumen, which is surrounded by a coil spring;
a stiffening tube coupled to the flexible guidewire lumen; and
one or more puller wires, which are coupled to the distal section and configured to bend the flexible guidewire lumen, thereby deflecting the expandable balloon.
